(19) United States
(12) Patent Application Publication
(10) Pub. No.: US 2006/0135546 A1
(43) Pub. Date: Jun. 22, 2006

(54) Methods for the purification of 20(S)- camptothecin

(76) Inventors: Jagadevappa Basavaraja, Yelahanka New Town (IN); Sudhir B. Chellappa, Sadashivanagar (IN); Vijay K. Shastry, Matunga (IN); Rao G.S.R. Subba, Malleshwaram (IN)

Correspondence Address: CLARK & ELBING LLP, 101 FEDERAL STREET, BOSTON, MA, 02110, US

(21) Appl. No.: 11/013,856

(22) Filed: Dec. 16, 2004

(51) Int. Cl. C07D 519/04; A61K 31/4745

(52) U.S. Cl. 514/283; 546/51

(57) ABSTRACT

The invention features scalable methods for the purification of (20S)-camptothecin.

### BACKGROUND OF THE INVENTION

The invention relates to methods for the purification of 20(S)-camptothecin (CPT).

CPT is a natural alkaloid plant product originally isolated from Camptothera acuminata. CPT and CPT derivatives, such as topecatan and irinotecan, are known to inhibit topoisomerase I. This property makes such compounds useful therapeutics for the treatment of neoplastic disorders.

The commercial synthesis of CPT derivatives begins with CPT, a starting material extracted from plants. The natural plant material, regardless of its source, does not contain only CPT, but also other natural products, including related alkaloid impurities, which have to be separated from CPT. For example, the plant material may also contain 10-hydroxy-20(S)-CPT, 10-methoxy-20(S)-CPT, 11-hydroxy-20(S)-CPT, 11-methoxy-20(S)-CPT, 20-deoxycamptothecin, and/or 9-methoxy-20(S)-CPT.

These naturally occurring impurities can be toxic, or can result in the formation of toxic side products when included in a process for the manufacture of a CPT derivative from CPT. Such problems can be avoided by extensively purifying CPT prior to its use in the semisynthesis of a CPT derivative.

CPT has become a valuable commercial commodity because of its use as a starting material for the preparation of an entire class of pharmaceutical agents. There is a need for efficient methods of purifying CPT on a commercial scale.

### SUMMARY OF THE INVENTION

The invention provides scalable and efficient methods for the purification of camptothecin. The methods of the present invention can be effective for producing camptothecin of 98% purity or higher.

The invention features a method of producing enriched camptothecin from crude camptothecin by solid liquid extraction. The method includes the steps of

(a) combining solid crude camptothecin with a liquid in which camptothecin is insoluble or sparingly soluble to form a mixture containing dissolved impurities; (b) separating the undissolved material from the liquid; and (c) repeating steps (a) and (b) until the undissolved material is enriched camptothecin.

In one embodiment, the mixture of step (a) is not heated. Desirably, the mixture is not heated above 70° C., 60° C., 50° C., or 46° C. Desirably, the volume of the mixture of step (a) is not reduced.

The invention further features a method of producing material containing 98% or greater camptothecin from enriched camptothecin by (a) combining the enriched camptothecin with glacial acetic acid; (b) heating the combination of step (a) to produce a solution having camptothecin dissolved therein; (c) cooling said solution to form a precipitate from dissolved camptothecin; and (d) separating the precipitate from the solution, wherein the precipitate contains 98% or greater camptothecin. Desirably, the precipitate contains at least 98.2%, 98.4%, 98.6%, 98.8%, or 99% camptothecin.

In one embodiment, at step (b) all of the enriched camptothecin is dissolved. Desirably, the method is performed using at least 15 g, 25 g, 50 g, 75 g, 100 g, or 125 g of enriched camptothecin.

The invention features a method of producing material containing 98% or greater camptothecin from enriched camptothecin by (a) loading enriched camptothecin onto a column packed with an adsorbent material; (b) passing a mobile phase across the adsorbent material; (c) collecting the eluent in fractions; and (d) combining camptothecin rich fractions and separating the dissolved solids therein to produce a material containing 98% or greater camptothecin. Desirably, the mobile phase is selected from chloroform and chloroform/acetone mixtures and the adsorbant material is silica.

The invention also features a method of purifying crude camptothecin by (a) combining solid crude camptothecin with a liquid in which camptothecin is insoluble or sparingly soluble to form a mixture containing dissolved impurities;

(b) separating the undissolved material from the liquid; (c) repeating steps (a) and (b) until the undissolved material is enriched camptothecin; and (d) recrystallizing the enriched camptothecin from a solvent to form a precipitate, wherein the precipitate contains 98% or greater camptothecin.

The recrystallization of step (d) can be performed using N,N-dimethylformamide, N-methylpyrrolidone, N,N-dimethylacetamide, acetic acid, hydrochloric acid, trifluoroacetic acid, dimethylsulfoxide, ethanol, and a mixtures thereof. Preferably, the recrystallization is performed using glacial acetic acid.

The invention features a method of purifying crude camptothecin, by (a) combining solid crude camptothecin with a liquid in which camptothecin is insoluble or sparingly soluble to form a mixture containing dissolved impurities; (b) separating the undissolved material from the liquid; (c) repeating steps (a) and (b) until said undissolved material is enriched camptothecin; (d) loading enriched camptothecin onto a column packed with an adsorbent material; (e) passing a mobile phase across the adsorbent material; (f) collecting the eluent in fractions; and (g) combining camptothecin rich fractions and separating the dissolved solids therein to produce a material containing 98% or greater camptothecin.

The mobile phase in the above method can be selected from chloroform, methylene chloride, diethyl ether, hexane, pentane, chloroform, acetone, methyl isobutyl ketone, methyl butyl ketone, methyl ethyl ketone, and mixtures thereof. Desirably, the mobile phase is selected from chloroform and chloroform/acetone mixtures.

The adsorbant material is in the above method can be selected from silica adsorbents, alumina adsorbents, magnesium silicate adsorbents, magnesia adsorbents, carbon adsorbents, diatomaceous earth adsorbents, and mixtures thereof. Desirably, the adsorbant material is silica.

In any of the methods of the invention including a solid-liquid extraction step, the method can be performed using at least 25 g, 50 g, 75 g, 100 g, 125 g, 150 g, 250 g, or even 500 g of crude camptothecin.

In any of the methods of the invention including a solid-liquid extraction step, the liquid can be selected from chloroform, acetone, methyl isobutyl ketone, methyl butyl ketone, methyl ethyl ketone, dimethylsulfoxide, and mixtures thereof. Desirably, the liquid is selected from chloroform, acetone, dimethylsulfoxide, and mixtures thereof.

In any of the methods of the invention including the purification of crude camptothecin, the crude camptothecin contains between 50% and 80% camptothecin. Desirably, the crude camptothecin contains from 55% to 80%, 60% to 80%, 65% to 80%, 65% to 75%, or 70% to 80% camptothecin.

In any of the methods of the invention including the purification of enriched camptothecin, the enriched camptothecin contains between 90% and 96% camptothecin. Desirably, the enriched camptothecin contains from 91% to 96%, 92% to 96%, 90% to 95%, 91% to 95%, 92% to 95%, 90% to 94%, or 91% to 94% camptothecin.

In any of the methods of the invention, the recovery of the camptothecin is at least 74%, 76%, 78%, 80%, 82%, 84%, 85%, 86%, 87%, 88%, 89%, 90%, 91%, 92%, 93%, or 94%.

The invention features a method of synthesizing a camptothecin derivative from camptothecin extracted from a plant by (a) purifying the camptothecin according to any of the methods of the invention; and (b) using the purified camptothecin of step (a) in the synthesis of said camptothecin derivative. Desirably the camptothecin derivative is irinotecan, topotecan, rubitecan, exatecan, lurtotecan, silatecan, 9-aminocamptothecin, afeletecan, gimatecan, PNU-166148, prothecan, CKD-602, karenitecin, SN-38, DRF-1042, or diflomotecan.

As used herein, “recovery” refers to the amount of CPT present (e.g., the purity times the mass) in the end product of the purification process in comparison to the starting material.

By “sparingly soluble” is meant that the solubility of CPT in a liquid at 25° C. is 1 g to 10 g per liter of liquid.

By “insoluble” is meant that the solubility of CPT in a liquid at 25° C. is less than 1 g per liter of liquid.

All references to purity or percentages of CPT in a mixture refer to the purity or percentage as determined using the assay of Example 6 on a dried solid (e.g., a solid from which volatiles have been removed by heating or under vacuum). The calculated purity or percentage accounts for the presence of alkaloid impurities, among other impurities.

By “enriched camptothecin” is meant material extracted from a plant and containing from 90% to 96% CPT. Enriched camptothecin can be prepared as described in Examples 1-3.

By “crude camptothecin” is meant material extracted from a plant and containing from 50% to 80% CPT. Crude camptothecin can be purchased commercially from Zhejiang Yixin Pharmaceutical Co., Ltd. (Zhejiang, PRC).

As used herein, “camptothecin derivatives” include irinotecan (CAMPTOSAR™; Yakult Honsha K K), topotecan (Hycamtin™; GSK), rubitecan (9-NC; SuperGen), exatecan (DX-8951f; Daiichi), lurtotecan (OSI-211; OSI), silatecan (DB67), 9-aminocamptothecin (IDEC-132; IDEC Pharmaceuticals), afeletecan (BAY 38-3441 a camptothecin glycoconjugate; Bayer AG), gimatecan (ST1481; Sigma-Tau Healthsci SpA), PNU-166148 (polymethacrylamide-CPT conjugate; Pharmacia), prothecan (PEG-CPT conjugate; Enzon Inc.), CKD-602 ((20S)-7-(2-isopropylamino)ethylcamptothecin; Chong Kun Dang Pharmaceutical Corp.), karenitecin (7-[(2-trimethylsilyl)ethyl]-20(S)-camptothecin; Bionumerik), 7-ethyl-10-hydroxycamptothecin (SN-38), DRF-1042 (Dr. Reddys Research Foundation, see J. Clin. Pharmacol. 44:723 (2004)), and homocamptothecins, such as diflomotecan (BN-80915; Roche). Camptothecin derivatives further include the compounds disclosed in U.S. Pat. Nos. 6,214,836; 6,177,439; 6,043,367; 6,040,313; 5,972,955; 5,932,588; 5,916,896; 5,889,017; 5,801,167; 5,731,316; 5,674,874; 5,658,920; 5,646,159; 5,633,260; 5,604,233; 5,597,829; 5,552,156; 5,552,154; 5,541,327; 5,525,731; 5,468,754; 5,447,936; 5,446,047; 5,401,747; 5,391,745; 5,364,858; 5,340,817; 5,244,903; 5,227,380; 5,225,404; 5,180,722; 5,122,606; 5,122,526; 5,106,742; 5,061,800; 5,053,512; 5,049,668; 5,004,758; 4,981,968; 4,943,579; 4,939,255; 4,894,456; and 4,604,463; and PCT Publication Nos. WO98/07727; WO96/39143; WO97/00876; WO98/28305; WO98/28304; WO95/22549; WO96/37496; WO97/28164; WO97/25332; WO98/35969; WO97/46564; WO97/46563; WO97/46562; WO96/34003(1996); WO96/02546; WO96/31532; WO98/14459; WO98/14468; WO98/15573; WO98/51703; WO98/07713; WO96/23794; WO95/10304; and WO98/36776, each of which is incorporated herein by reference.

As used herein, “alkaloid impurities” refers to any one or more of 10-hydroxy-20(S)-CPT, 10-methoxy-20(S)-CPT, 11-hydroxy-20(S)-CPT, 11-methoxy-20(S)-CPT, 20-deoxycamptothecin, and 9-methoxy-20(S)-CPT.

For any reference provided herein to either rings A-E or a numbered position in CPT or a CPT derivative, the recited position is defined by the scheme below.

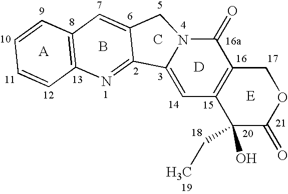

The methods of the invention provide for the efficient commercial scale production of highly purified CPT.

Other features and advantages of the invention will be apparent from the following Detailed Description and the claims.

### DETAILED DESCRIPTION

The invention features scalable and efficient methods for the purification of CPT. The methods remove impurities from a starting material used in the semisynthesis of camptothecin derivatives. These impurities can complicate the synthesis of a camptothecin derivative or be difficult and costly to remove from the final product.

(20S)-Camptothecin

CPT has a pentacyclic structure, shown below.

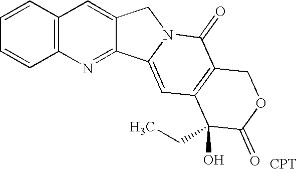

CPT is an alkaloid originally isolated from the Chinese tree Camptotheca acuminata. The majority of CPT is produced in the People's Republic of China. CPT can also be extracted from Nothapodytes phoetida, a tree native to the Indian subcontinent.

The extraction of CPT from plant material typically involves drying and grinding the plant material, followed by hot extraction using an organic solvent (e.g., using Soxhlet extractor). Removal of the solvent yields the crude extract of the plant material. CPT obtained from plant extracts generally has a purity of 50% or greater. Crude extracts containing from 50% to 80% CPT are commercially available. This material, crude camptothecin, can be purified as described in Examples 1-5 to produce material which is a suitable starting material for the synthesis of CPT derived pharmaceuticals.

Camptothecin Derivatives

Camptothecin derivatives can be prepared using semisynthetic processes that utilize CPT as a starting material. These semisynthetic protocols are described, for example, in U.S. Pat. No. 5,004,758 (topotecan); U.S. Pat. No. 4,604,463 (irinotecan); Lavergne et al., Bioorg. Med. Chem. Lett., 7: 2235 (1997) (homocamptothecins); Kaskar et al., Bioorg. Med. Chem. 11:451 (2003) (silatecan);. Lerchen et al., J. Med. Chem. 44:4186 (2001) (afeletecan); Dallavalle et al., Bioorg. Med. Chem. Lett. 11:291 (2001) (gimatecan); Luzzio et al., J. Med. Chem. 38:395 (1995) (lurtotecan); Wall et al., J. Med. Chem. 36:2689 (1993) (9-aminocamptothecin); and Conover et al., Cancer Chemother. Pharmacol. 42:407 (1998) (prothecan). Further synthetic protocols utilizing CPT can be found in U.S. Pat. Nos. 6,214,836; 6,177,439; 6,043,367; 6,040,313; 5,972,955; 5,932,588; 5,916,896; 5,889,017; 5,801,167; 5,731,316; 5,674,874; 5,658,920; 5,646,159; 5,633,260; 5,604,233; 5,597,829; 5,552,156; 5,552,154; 5,541,327; 5,525,731; 5,468,754; 5,447,936; 5,446,047; 5,401,747; 5,391,745; 5,364,858; 5,340,817; 5,244,903; 5,227,380; 5,225,404; 5,180,722; 5,122,606; 5,122,526; 5,106,742; 5,061,800; 5,053,512; 5,049,668; 4,981,968; 4,943,579; 4,939,255; and 4,894,456; and PCT Publication Nos. WO98/07727; WO96/39143; WO97/00876; WO98/28305; WO98/28304; WO95/22549; WO96/37496; WO97/28164; WO97/25332; WO98/35969; WO97/46564; WO97/46563; WO97/46562; WO96/34003(1996); WO96/02546; WO96/31532; WO98/14459; WO98/14468; WO98/15573; WO98/51703; WO98/07713; WO96/23794; WO95/10304; and WO98/36776. Each of the patents and patent applications above is incorporated herein by reference.

Purification by Solid-Liquid Extraction

Crude camptothecin (50-80% CPT) can be purified to enriched camptothecin (90-96% CPT) using the method described in Examples 1-3. The method is a solid-liquid extraction in which the impurities, more soluble than the CPT in the liquid, are extracted from the crude camptothecin solid. Liquids are selected in which the CPT is sparingly soluble or insoluble. Liquids which can be used in the method include chloroform, acetone, methyl isobutyl ketone, methyl butyl ketone, methyl ethyl ketone, dimethylsulfoxide, and mixtures thereof.

To assist the extraction of impurities, the mixture of crude camptothecin is stirred. Optionally, the method includes sonication or gentle heating to assist in the extraction of the impurities.

Purification by Recrystallization

Enriched camptothecin can be further purified by recrystallization as described, for example, in Example 4. Preferred amounts of the solvent to be employed in the recrystallization are from about 10 to 50 volumes of solvent per volume of camptothecin, depending upon the solvent used. Solvents which may be employed in the recrystallization include N,N-dimethylformamide, N-methylpyrrolidone, N,N-dimethylacetamide, acetic acid, hydrochloric acid, trifluoroacetic acid, dimethylsulfoxide, ethanol, and a mixtures thereof. Desirably, the recrystallization is performed using glacial acetic acid.

The camptothecin can be dissolved by heating the solvent as needed to dissolve most or all of the camptothecin material, e.g., to the point of reflux. Cooling the saturated solution results in the crystallization of camptothecin.

Purification by Chromatography

Enriched camptothecin can be further purified using chromatographic methods as described in Example 5. For example, a column, such as columns made by Ace Glass or Fisher Scientific, is charged with a solvent along with an adsorbent material, e.g., a silica, silica gel., alumina, magnesium silicate, magnesia, carbon, diatomaceous earth, porous beads, and special adsorbents or a combination thereof obtained for example from Aldrich or Sigma. The adsorbent, e.g. silica or alumina, which is used generally has a size of 70-300 mesh. Other grades may also be used. Any column may be used which allows the adsorbent to be packed inside the column (composed of plastics, Pyrex™ or other forms or glass or glass-like materials, pure metals or suitable alloys of metals or alloys of metals with nonmetals, steel or stainless steel or a suitable combination thereof known and understood by those skilled in the art) in a fashion such that the sample to be chromatographed may be introduced at one end of the adsorbent and then separated into one or more individual components and eluting through the other end of the column by flowing solvent through the packed adsorbent. This process may be carried out at low pressures (less than one atmosphere) up to very high pressures of several thousand atmospheres of pressure known to those skilled in the art, including, but not limited to, HPLC. The separation may also be carried out at normal room temperature or higher for example, 100° C., or lower for example, 0° C., temperatures.

The mobile phase for the chromatographic purification can be selected from chloroform, methylene chloride, diethyl ether, hexane, pentane, chloroform, acetone, methyl isobutyl ketone, methyl butyl ketone, methyl ethyl ketone, and mixtures thereof. Desirably, the mobile phase is chloroform and chloroform/acetone mixtures.

The eluent from the column can be monitored using any of a variety of methods, including thin layer chromatography or the HPLC assay of Example 6.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

In the examples below, crude CPT is 70% CPT isolated from the Chinese tree Camptotheca accuminata. This material was purchased from a commercial supplier.

### EXAMPLE 1

### Enrichment of Crude CPT Using Dimethylsulfoxide and Chloroform

1,500 mL of dimethylsulfoxide and 100 g of crude CPT were charged into round bottomed flask. The mixture was stirred for 30 minutes at 42 to 45° C. To the mixture was added 2,000 mL of chloroform. Stirring was continued for another 30 minutes at same temperature (42-45° C.). The mixture was allowed to cool to room temperature. The undissolved solid was isolated by filtration. The undissolved solid was then twice subjected to the same process mentioned above twice and the resultant product, isolated by filtration, was washed with water (500 mL) followed by acetone (250 mL) and dried under vacuum at 50° C. The process yielded 67 g of enriched material containing 93% CPT. The recovery of the starting CPT was 90% or greater.

The CPT remaining in the filtrate was recovered by combining all of the filtrates, reducing the volume by distillation to remove the chloroform, and adding water to precipitate the dissolved CPT. The solid was filtered, washed with water followed by acetone and dried to obtain 7.5 g of material containing 75% CPT.

### EXAMPLE 2

### Enrichment of Crude CPT Using Acetone

3,000 mL of acetone and 100 g of crude CPT were charged into a round bottomed flask. The mixture was stirred for 30 minutes at 42 to 45° C. and then was allowed to cool to room temperature. The undissolved solid was isolated by filtration. The undissolved solid was then twice subjected to the same process mentioned above twice and the resultant product, isolated by filtration, was dried at about 50° C. The process yielded 66 g of enriched material containing 92% CPT. The recovery of the starting CPT was 95% or greater.

The CPT remaining in the filtrate was recovered by combining all of the filtrates and reducing the volume by distillation. The resulting solid yielded 14 g of material containing 50% CPT.

### EXAMPLE 3

### Enrichment of Crude CPT Using Dimethylsulfoxide and Acetone

1,000 mL of dimethylsulfoxide and 100 g of crude CPT were charged into round bottomed flask. The mixture was stirred for 30 minutes at ambient temperature. To the mixture was added 500 mL acetone. Stirring was continued for another 30 minutes at ambient temperature. The undissolved solid was isolated by filtration. The undissolved solid was then twice subjected to the same process described above and the resultant product, isolated by filtration, was washed with water (500 mL) followed by acetone (250 mL) and dried under vacuum at 50° C. The process yielded 65 g of enriched material containing 96% CPT. The recovery of the starting CPT was 90%.

The CPT remaining in the filtrate was recovered by combining all of the filtrates, reducing the volume by distillation to remove the acetone, and adding water to precipitate the dissolved CPT. The solid was filtered, washed with water followed by acetone and dried to obtain 9 g of material containing 65% CPT.

### EXAMPLE 4

### Purification of Enriched CPT by Recrystallization from Glacial Acetic Acid

750 mL of glacial acetic acid and 25 g of enriched CPT (assay 92% to 94%) were charged into round bottomed flask. The mixture was stirred for 2 hours at 100° C. to dissolve all of the solids. The solution was then allowed to cool to room temperature overnight, resulting in the formation of a precipitate. The precipitate was filtered and washed with water (100 mL) followed by methanol (50 mL) and dried under vacuum at 50° C. The resulting product weighed 22.54 g and contained 99.10% CPT.

The CPT remaining in the filtrate was recovered by concentrating the acetic acid solution to about 50 mL and cooling the solution to ambient temperature to produce a second precipitate. The precipitate was isolated by filtration to yield 1.53 g of material containing 80.5% CPT.

### EXAMPLE 5

### Purification of Enriched CPT by Means of Chromatography

2.5 g enriched CPT from Example 2, 95% purity, was adsorbed to 75 g silica (mesh size 100-200) using a combination of 150 mL of chloroform and acetone in the ratio 8:2. This mixture was stirred at room temperature for 30 minutes in order to achieve selective adsorption of CPT on the adsorbant. The above adsorbed sample was applied to the column (Borosil glass, 2″×24″) packed using 450 g silica and 1,000 mL of Chloroform as a slurry. Then the components of the enriched CPT were separated and eluted by using combinations of chloroform and acetone as a mobile phase. Initially, 100% chloroform was used followed by chloroform:acetone in the ratios of 8:2, 7:3, 6:4, 5:5 and 4:6. The fractions were collected and analyzed for CPT using Hewlitt Packard Make HPLC 1100 series. The first two fractions contained most of the impurities. CPT of purity 98%-99% was obtained in the fractions 3 and 4. The final yield was not less than 90% of input feed to the column.

### EXAMPLE 6

### CPT Assay

The purity of CPT-containing samples were determined by HPLC of the sample and a reference standard. First, a CPT sample was prepared and characterized using a NMR spectra. The identified peaks were interpreted quantitatively and were found to be 99% purity. For all calculations of CPT purity, the absolute purity of the reference standard was taken as 99%. The test sample and the reference sample were subjected to HPLC analysis, 1100 Series Hewlett Packard Binary Pump HPLC, under the following conditions: column: C 18, BDS Hypersil (250×4.6 mm, 5 m); mobile phase: water/acetonitrile (time) 75/25 (0.0), 70/30 (5.0), 60/40 (10.0), 45/55 (15.0), 40/60 (20.0), 75/25 (25.0); flow rate: 1.0 mL/minute; wave length: 254 nm; temperature: room temperature.

The test sample and reference sample were prepared by dissolving 25 mg of the sample in 50 mL dimethylsulfoxide. The test sample purity was calculated using the following equation:
% Purity=(Area of Sample/Area of Std)×(Dilution of Std/Dilution of sample)×Purity of Std.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of producing enriched camptothecin from crude camptothecin, said method comprising:
a) combining solid crude camptothecin with a liquid in which camptothecin is insoluble or sparingly soluble to form a mixture containing dissolved impurities;
b) separating the undissolved material from the liquid; and
c) repeating steps (a) and (b) until said undissolved material is enriched camptothecin.

2. The method of claim 1, wherein the mixture of step (a) is not heated above 50° C.

3. The method of claim 1, wherein the volume of the mixture of step (a) is not reduced.

4. The method of claim 1, wherein said liquid is selected from chloroform, acetone, methyl isobutyl ketone, methyl butyl ketone, methyl ethyl ketone, dimethylsulfoxide, and mixtures thereof.

5. The method of claim 1, wherein said crude camptothecin contains between 65% and 75% camptothecin.

6. The method of claim 1, wherein said enriched camptothecin contains between 92% and 96% camptothecin.

7. The method of claim 1, wherein the recovery of the camptothecin is 85% or greater.

8. A method of producing material containing 98% or greater camptothecin from enriched camptothecin, said method comprising:
a) combining said enriched camptothecin with glacial acetic acid;
b) heating the combination of step (a) to produce a solution having camptothecin dissolved therein;
c) cooling said solution to form a precipitate from dissolved camptothecin; and
d) separating said precipitate from said solution, wherein said precipitate contains 98% or greater camptothecin.

9. The method of claim 8, wherein said enriched camptothecin contains between 92% and 96% camptothecin.

10. The method of claim 8, wherein at step (b) all of the enriched camptothecin is dissolved.

11. The method of claim 8, wherein said precipitate contains 99% or greater camptothecin.

12. The method of claim 8, wherein the mixture of step (a) comprises at least 25 g of enriched camptothecin.

13. The method of claim 8, wherein the recovery of the camptothecin is 85% or greater.

14. A method of producing material containing 98% or greater camptothecin from enriched camptothecin, said method comprising:
a) loading enriched camptothecin onto a column packed with an adsorbent material;
b) passing a mobile phase of chloroform followed by chloroform/acetone mixtures across said adsorbent material;
c) collecting the eluent in fractions; and
d) combining camptothecin rich fractions and separating the dissolved solids therein to produce a material containing 98% or greater camptothecin.

15. The method of claim 14, wherein said adsorbant material is silica.

16. The method of claim 14, wherein the recovery of the camptothecin is 85% or greater.

17. A method of purifying crude camptothecin, said method comprising:
a) combining solid crude camptothecin with a liquid in which camptothecin is insoluble or sparingly soluble to form a mixture containing dissolved impurities;
b) separating the undissolved material from the liquid;
c) repeating steps (a) and (b) until said undissolved material is enriched camptothecin; and
d) recrystallizing said enriched camptothecin from a solvent to form a precipitate, wherein said precipitate contains 98% or greater camptothecin.

18. A method of purifying crude camptothecin, said method comprising:
a) combining solid crude camptothecin with a liquid in which camptothecin is insoluble or sparingly soluble to form a mixture containing dissolved impurities;
b) separating the undissolved material from the liquid;
c) repeating steps (a) and (b) until said undissolved material is enriched camptothecin;
d) loading enriched camptothecin onto a column packed with an adsorbent material;
e) passing a mobile phase across said adsorbent material;
f) collecting the eluent in fractions; and
g) combining camptothecin rich fractions and separating the dissolved solids therein to produce a material containing 98% or greater camptothecin.

19. A method of synthesizing a camptothecin derivative from camptothecin extracted from a plant, said method comprising the steps of:
a) purifying said camptothecin according to any of claims 1, 2, 8, 14, 16, 17, or 18; and
b) using the purified camptothecin of step (a) in the synthesis of said camptothecin derivative.

20. The method of claim 19, wherein said camptothecin derivative is irinotecan, topotecan, rubitecan, exatecan, lurtotecan, silatecan, 9-aminocamptothecin, afeletecan, gimatecan, PNU-166148, prothecan, CKD-602, karenitecin, SN-38, DRF-1042, or diflomotecan.
